(19) United States
(12) Patent Application Publication
(10) Pub. No.: US 2018/0222909 A1
(43) Pub. Date: Aug. 9, 2018

(54) PROCESS FOR THE PREPARATION OF IBRUTINIB AND NEW SYNTHESIS INTERMEDIATE

(71) Applicant: OLON S.P.A., Rodano (IT)

(72) Inventors: Giorgio BERTOLINI, Rodano (IT); Ilaria FERRANDO, Lugano (CH); Mara SADA, Rodano (IT)

(21) Appl. No.: 15/747,657

(22) PCT Filed: Jul. 27, 2016
(86) PCT No.: PCT/IB2016/054476

§ 371 Date: Jan. 25, 2018

(30) Foreign Application Priority Data
Jul. 29, 2015 (IT) UB2015A002576

(51) Int. Cl. C07D 487/04

(52) U.S. Cl. CPC C07D 487/04

(57) ABSTRACT

Subject-matter of the invention is a process for the preparation of ibrutinib and intermediate compound.

### TECHNICAL FIELD

Ibrutinib is an antitumoral compound, currently used in the therapy of some lymphomas. Its International Nonproprietary Name (INN) is 1-[(3R)-3-[4-amino-3-(4-phenoxyphenyl)-1H-pyrazolo[3,4-d]pyrimidin-1-yl]piperidin-1-yl]prop-2-en-1-one and has the following structural formula:

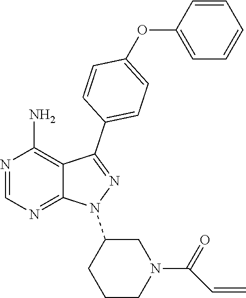

Several syntheses are known for the preparation of ibrutinib.

WO2008/039218 describes a preparation of ibrutinib involving a Suzuki reaction on 3-iodo-1H-pyrazolo[3,4-d]pyrimidin-4-amine to introduce the phenoxyphenyl group and later a Mitsunobu reaction to introduce the piperidine ring. In both cases, the obtained compounds are not of particularly high purity, as demonstrated by the fact that purifications by chromatography are required.

It is known that, in the large scale industrial synthesis, each reaction and purification step is expensive and requires complex procedures and it is also known that the purification of intermediate and final compounds, by means of chromatographic techniques, is neither particularly feasible nor economically convenient when carried out on a large scale and large amounts of product.

There is therefore the need of finding new alternative synthesis routes that allow to obtain ibrutinib with good purity and good yields, thus avoiding at the same time the need of employing expensive procedures which are scarcely suitable for industrial applications.

### OBJECTS OF THE INVENTION

It is an object of the invention to provide an alternative synthesis of ibrutinib that allows to avoid the drawbacks of the prior art.

It is a further object of the invention to provide a synthesis of ibrutinib which is suitable for the industrial use and economically convenient.

It is a further object of the invention to provide a new synthesis intermediate, which is useful in the process for the preparation of ibrutinib and also of other molecules.

### DESCRIPTION OF THE INVENTION

According to one of its aspects, the invention relates to a process for the preparation of ibrutinib of formula (I)

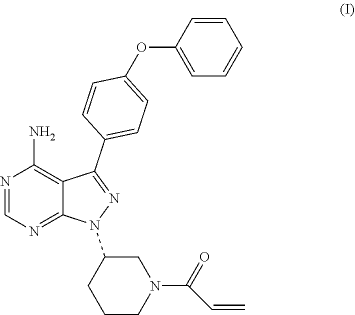

comprising the following steps:
a. carrying out a Mitsunobu reaction by reacting 3-bromo-1H-pyrazolo[3,4-d]pyrimidin-4-amine of formula (II)

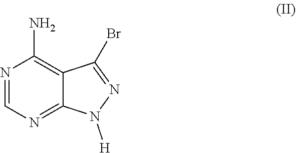 [SMILES: Nc1ncnc2[nH]nc(Br)c12]

with the protected piperidine of formula (III)

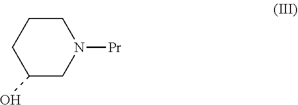

wherein Pr is a protecting group, and further reacting the obtained product with an acidic solution in which the acid is selected from an inorganic acid and trifluoroacetic acid,
b. carrying out a Suzuki reaction on the so obtained compound of formula (IV)

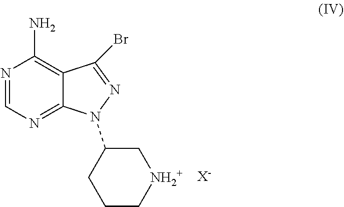

wherein X⁻ is the anionic residue of an acid selected from inorganic acids and trifluoroacetic acid, with 4-phenoxyphenylboronic acid, and then reacting the obtained product with an acidic solution in which the acid is selected from an inorganic acid and trifluoroacetic acid, and
c. reacting the so obtained compound of formula (V)

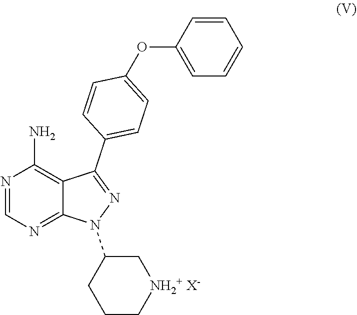

wherein X⁻ is as defined above, with acryloyl chloride to obtain ibrutinib of formula (I).

In the present description, Pr group is a protecting group that can be removed by hydrolysis, in particular by acid hydrolysis. As it will be shown, in this way, from the reaction of step (a), the compound of formula (III) is obtained that can be directly reacted without the need of intermediate purifications.

A preferred Pr group according to the invention is the tert-butyloxycarbonyl group. The reaction of step (a) is carried out according to the known Mitsunobu reaction, in an organic solvent, advantageously tetrahydrofuran (THF), in presence of a phosphine selected from, for example, triphenylphosphine, diethylphenylphosphine, dicyclohexylphenylphosphine, tri-tert-butylphosphine, trihexylphosphine and tricyclohexylphosphine, the triphenylphosphine being preferred, and of an azodicarboxylate such as for example dimethyl azodicarboxylate, diethyl azodicarboxylate, diisopropyl azodicarboxylate (DIAD), dibenzyl azodicarboxylate, di-tert-butyl azodicarboxylate, the diisopropyl 1,2-hydrazinedicarboxylate (DIAD) being preferred.

The reaction is preferably carried out at room temperature for a period comprised between 3 and 24 hrs, advantageously about 10-12 hrs.

The reaction mixture is then treated with acids, advantageously with an acid selected from hydrochloric acid, hydrobromic acid, trifluoroacetic acid in water, to obtain the corresponding salt of the compound of formula (IV), that can be directly used in the reaction of the next step.

Hydrochloric acid is particularly preferred in the preparation of the salt of step (a). The salification of the product obtained from step (a) is particularly convenient since it allows to obtain the compound with a good purity. Of course this salification is only possible because the amine group of piperidine is deprotected. In addition, if desired, the so obtained salt is easily crystallized. On the contrary, if the amine group of piperidine is kept protected, for example with BOC, an oil would be obtained which could be purified only by means of methods less suitable for the industrial use, such as for example the chromatography.

The reaction of step (b) is carried out according to the methods of the Suzuki reaction, involving the presence of a solvent or a solvent mixture, e.g. water and an alcohol, e.g. ethanol, as well as dioxane, tetrahydrofuran, dimethylformamide, toluene, alcohols, acetone and acetonitrile.

The reaction involves in addition a base, e.g. a hydroxide, a phosphate, a carbonate, the sodium or potassium tert-butoxide, an acetate or triethylamine, the sodium or potassium carbonate being preferred, and a suitable catalyst.

As a catalyst it is possible to use, for example, a palladium catalyst such as tetrakis(triphenylphosphine)palladium as well as palladium with different phosphine ligands; said compounds can be synthesized in advance or prepared in situ, according to known techniques. For example, palladium acetate or other palladium complexes can be used together with free phosphines which generate in situ the desired catalyst, as it is known by the person skilled in the field.

Advantageously, the reaction is carried out in an inert environment, e.g. under argon. The reaction is preferably carried out under reflux for a period comprised between 3 and 24 hrs, advantageously about 10-12 hrs.

The reaction mixture is then treated with acids, as described above for step (a), for example with an acid selected from hydrochloric acid, hydrobromic acid, trifluoroacetic acid in water, to obtain the corresponding salt of the compound of formula (V), that can be directly used in the reaction of the next step. Advantageously, hydrochloric acid is used to obtain the corresponding salt of the compound of formula (V), that can be directly used in the reaction of the next step. Also in this case, the preparation and use of the salt leads to the advantages described above with respect to the use of the compound with the nitrogen of piperidine protected.

The reaction of step (c) involves the formation of the amide group and can be carried out with different known techniques for the formation of amides. Such reaction is known in the art, as well for the preparation of ibrutinib, and the person skilled in the field can select the most convenient method. However, an example of such reaction is provided in the following experimental section.

Ibrutinib can be isolated from the reaction mixture according to conventional procedures and, if desired or needed, purified. However, by working with the process of the invention, ibrutinib of very high purity is obtained without the need of final purifications, having a purity exceeding 99% (HPLC).

Detailed examples of preparation are provided in the experimental section of the present description.

The compound of formula (IV′)

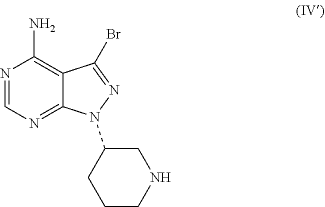

and the salt thereof, in particular the hydrochloride salt, is a new compound and represents a further object of the present invention.

As it can be noted, the Suzuki reaction is carried out on the compound of formula (IV) wherein the piperidine nitrogen is not protected. Nevertheless undesired reactions do not occur and the compound of formula (V) is obtained with good yields and excellent purity.

As it will be possible to observe from the description of the following examples as well, the process of the invention is completely feasible on large scale, because it does not require any step which is too laborious or not possible on industrial scale, such as the chromatographic purification.

The starting compound of formula (II) can be easily obtained by reacting 1H-pyrazolo[3,4-d]pyrimidin-4-amine and N-bromosuccinimide, according to the methods well known to the person skilled in the field. However, a detailed example of this reaction is provided in the experimental section.

The use of bromine instead of iodine in the process of the invention leads to the further advantage of being cost-effective, since N-bromosuccinimide is a less expensive reagent with respect to N-iodosuccinimide and this has clearly, in the large scale production, an important impact.

Moreover, although it is generally known that bromine is a less reactive species with respect to iodine in this kind of reactions, surprisingly the Applicant could observe that the obtained yields are comparable to those of the same reactions on the iodo-derived intermediates, and this is a further unexpected aspect of the process of the invention.

### EXPERIMENTAL SECTION

### Example 1

### Step (a)

3-Bromo-1H-pyrazolo[3,4-d]pyrimidin-4-amine (5.00 g, 23.36 mmol), (S)-3-hydroxy-N—BOC-piperidine (6.11 g, 30.37 mmol) and triphenylphosphine (9.19 g, 35.04 mmol) are suspended in anhydrous THF (250 ml). DIAD (6.9 ml, 35.0 mmol) is added in 4 hrs. The mixture was stirred overnight. Then the mixture was filtered and concentrated hydrochloric acid (25 ml) is added to the solution. The mixture was heated to 50° C. for 3 hrs and then cooled in an ice bath. The precipitated solid was filtered and washed with THF (2×10 ml). After drying under vacuum, the (R)-3-bromo-1-(piperidin-3-yl)-1H-pyrazolo[3,4-d]pyrimidin-4-amine hydrochloride was obtained as beige solid (5.17 g, 15.50 mmol, 66%, HPLC purity 98.3%).

### Example 2

### Step (b)

(R)-3-bromo-1-(piperidin-3-yl)-1H-pyrazolo[3,4-d]pyrimidin-4-amine (1.25 g, 3.75 mmol), 4-phenoxyphenylboronic acid (1.20 g, 5.62 mmol), potassium phosphate (3.98 g, 18.73 mmol) are suspended in water and ethanol (2/5, 34 ml) and degassed with argon for 15 minutes. Tetrakis(triphenylphosphine)palladium (216 mg, 0.19 mmol) is added and degassed for other 15 minutes. The mixture is left at reflux overnight. After cooling to room temperature, water (15 ml) is added and the pH is adjusted to 1 by adding dropwise hydrochloric acid (about 4.5 ml). The solid is removed by filtration and the solvent is concentrated. The aqueous phase is washed with ethyl acetate (4×5 ml). Dichloromethane (15 ml) is added and the pH is adjusted to 12 with an aqueous solution of KOH (1 g/ml). The aqueous phase is extracted with dichloromethane (2×5 ml). The combined organic phases are dried over MgSO₄ and the solvent is evaporated under reduced pressure. A raw product in the form of solid foam is obtained (610 mg, 1.56 mmol). The foam is dissolved in THF (6 ml) and then a 2 N hydrochloric acid solution in diethyl ether (about 4.0 ml) is added dropwise under vigorous stirring. The solid is isolated by filtration, washed with diethyl ether (2×5 ml) and dried under vacuum to give (R)-3-(4-phenoxyphenyl)-1-(piperidin-3-yl)-1H-pyrazolo[3,4-d]pyrimidin-4-amine hydrochloride (718 mg, 1.70 mmol, 45%, HPLC purity: 99.2%).

### Optional Purification

The obtained compound (570 mg, 1.28 mmol) has been dissolved in methanol (3.5 ml) and added dropwise to acetone (35 ml). The solid was filtered and dried under vacuum to give (R)-3-(4-phenoxyphenyl)-1-(piperidin-3-yl)-1H-pyrazolo[3,4-d]pyrimidin-4-amine hydrochloride (437 mg, 1.03 mmol, 81%, HPLC purity 99.7%).

### Example 3

### Step (c)

(R)-3-(4-phenoxyphenyl)-1-(piperidin-3-yl)-1H-pyrazolo[3,4-d]pyrimidin-4-amine hydrochloride (100 mg, 0.23 mmol) is suspended in anhydrous dichloromethane (2.0 ml). DBU (0.10 ml, 0.68 mmol) is added at 0° C. and then acryloyl chloride (about 15.5 μL, 0.19 mmol) in anhydrous dichloromethane (2.0 ml) is added as well at 0° C. in 1 hr. The solution is left to warm to room temperature and then left under stirring overnight. It is diluted with dichloromethane (7 ml) and washed with 1 M hydrochloric acid (2×2.5 ml), and then with a saturated solution of NaHCO₃ (2.5 ml) and water (2.5 ml). The solution is dried over MgSO₄ and the solvent evaporated to give (R)-1-(3-(4-amino-3-(4-phenoxyphenyl)-1H-pyrazolo[3,4-d]pyrimidin-1-yl)piperidin-1-yl)prop-2-en-1-one (77 mg, 0.18 mmol, 80%, HPLC purity 99.9%).

### Example 4

### Preparation of 3-bromo-1H-pyrazolo[3,4-d]pyrimidin-4-amine

1H-pyrazolo[3,4-d]pyrimidin-4-amine (20.0 g, 148.0 mmol) and N-bromosuccinimide (27.7 g, 155.4 mmol) are suspended in DMF (400 ml) and heated to 80° C. for 2.5 hrs. After cooling to room temperature, the reaction is quenched with water (dropwise, 800 ml). The precipitate is filtered and suspended in a saturated solution of Na₂SO₃ (100 ml). The solid was filtered and washed with water (3×100 ml) and cold ethanol (2×50 ml). After drying under vacuum, the 3-bromo-1H-pyrazolo[3,4-d]pyrimidin-4-amine is obtained as beige solid (22.7 g, 105.9 mmol, 72%).

1. A process for the preparation of ibrutinib of formula (I) 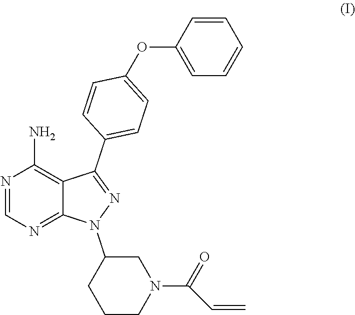 [SMILES: C=CC(=O)N1CCCC(n2nc(-c3ccc(Oc4ccccc4)cc3)c3c(N)ncnc32)C1]
comprising the following steps:
a. carrying out a Mitsunobu reaction by reacting 3-bromo-1H-pyrazolo[3,4-d]pyrimidin-4-amine of formula (II) 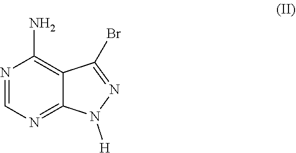 [SMILES: Nc1ncnc2[nH]nc(Br)c12]
with the protected piperidine of formula (III) 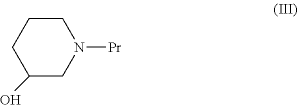
wherein Pr is a protecting group, and further reacting the obtained product with an acidic solution in which the acid is selected from an inorganic acid and trifluoroacetic acid,
b. carrying out a Suzuki reaction on the so obtained compound of formula (IV) 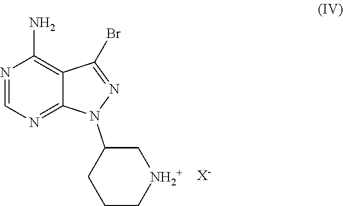
wherein X⁻ is the anionic residue of an acid selected from inorganic acids and trifluoroacetic acid, with 4-phenoxyphenylboronic acid, and then reacting the obtained product with an acidic solution in which the acid is selected from an inorganic acid and trifluoroacetic acid, and
c. reacting the so obtained compound of formula (V) 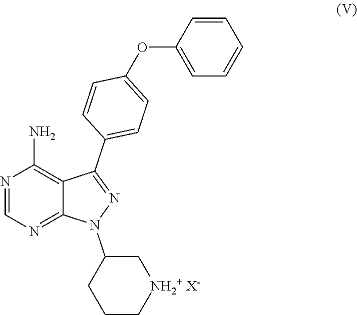
wherein X⁻ is as defined above, with acryloyl chloride to obtain ibrutinib of formula (I).

2. The process according to claim 1, wherein said Pr is a protecting group that can be removed by hydrolysis.

3. The process according to claim 1, wherein said Pr is a tert-butyloxycarbonyl group.

4. The process according to claim 1, wherein the reaction of step (a) is carried out in tetrahydrofuran (THF), in the presence of a phosphine and of an azodicarboxylate.

5. The process according to claim 4, wherein said azodicarboxylate is diisopropyl-1,2-hydrazinedicarboxylate.

6. The process according to claim 1, wherein the reaction of step (b) is carried out in presence of a mixture of water and alcohol, a base, and a palladium catalyst.

7. The process according to claim 6, wherein said catalyst is tetrakis(triphenylphosphine)palladium.

8. The process according to claim 1, wherein the starting compound of formula (II) is prepared by reacting 1H-pyrazolo[3,4-d]pyrimidin-4-amine and N-bromosuccinimide.

9. A compound of formula (IV′) 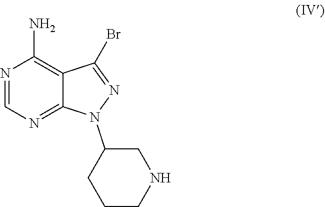 [SMILES: Nc1ncnc2c1c(Br)nn2C1CCCNC1]
or a salt thereof.

10. The compound according to claim 9, wherein said salt is selected from the group consisting of inorganic salts and trifluoroacetic acid

11. The compound according to claim 9, wherein said salt is a hydrochloride salt.

12. The process according to claim 2, wherein said hydrolysis comprises acid hydrolysis.

13. The process according to claim 2, wherein the reaction of step (a) is carried out in tetrahydrofuran (THF), in the presence of a phosphine and of an azodicarboxylate.

14. The process according to claim 3, wherein the reaction of step (a) is carried out in tetrahydrofuran (THF), in the presence of a phosphine and of an azodicarboxylate.

15. The process according to claim 2, wherein the reaction of step (b) is carried out in presence of a mixture of water and alcohol, a base, and a palladium catalyst.

16. The process according to claim 3, wherein the reaction of step (b) is carried out in presence of a mixture of water and alcohol, a base, and a palladium catalyst.
